(19) United States
(12) Patent Application Publication
(10) Pub. No.: US 2026/0133188 A1
(43) Pub. Date: May 14, 2026

(54) NANOBIOSENSORS AND METHODS FOR DETECTION AND MEASUREMENT USING NANOBIOSENSORS

(71) Applicant: The Trustees of Columbia University in the City of New York, New York, NY (US)

(72) Inventors: Qiao Lin, New York, NY (US); Milan Stojanovic, New York, NY (US); Shifeng Yu, New York, NY (US); Chao Su, New York, NY (US); Nenad Milosavic, New York, NY (US); Wenting Dai, New York, NY (US)

(21) Appl. No.: 19/296,613

(22) Filed: Aug. 11, 2025

Related U.S. Application Data
(60) Provisional application No. 63/681,501, filed on Aug. 9, 2024.

(51) Int. Cl. G01N 33/543; G01N 27/414; G01N 33/53; G01N 33/74

(52) U.S. Cl. CPC G01N 33/5438; G01N 27/4145; G01N 27/4146; G01N 33/5308; G01N 33/74; G01N 2410/04

(57) ABSTRACT

Disclosed is a device including a transistor that includes a graphene-based conducting channel, a functional layer modified with an aptamer, and attached to the graphene-based conducting channel via a polymer, such that the aptamer binds with a target biomarker. A method for manufacturing an aptamer-based biosensor, and a method for detecting a target biomarker using the aptamer-based biosensor, are also provided.

### CROSS-REFERENCE TO RELATED APPLICATIONS

This application claims the benefit under 35 U.S.C. § 119(e) of U.S. provisional application Ser. No. 63/681,501, filed on 9 Aug. 2024, which is herein incorporated by reference in its entirety.

### STATEMENT REGARDING FEDERALLY SPONSORED RESEARCH OR DEVELOPMENT

This invention was made with government support under DK126739 and GM138843 awarded by the National Institutes of Health. The government has certain rights in the invention.

### INCORPORATION OF SEQUENCE LISTING

A sequence listing containing the file named Columbia_88800730-000518NP_Sequence_ListingXML which is 2.043 bytes (measured in MS-Windows®) created on 6 Aug. 2025, and comprises one sequence, is incorporated herein by reference in its entirety.

### TECHNICAL FIELD

This disclosure relates to an aptamer-based graphene affinity nanosensor for detection of biomarkers, and a system and method for detecting biomarkers using an aptamer-based graphene affinity nanosensor.

### BACKGROUND

Graphene nanosensors, with small footprints, can be built on optically transparent and compliant substrates, and are hence ideally suited to measurement of biomarkers in physiological fluids Arginine vasopressin (AVP), an anti-diuretic hormone, is a clinically important low-molecular-weight biomarker (1084 Da) as well as a therapeutic molecule for severe hemorrhage, septic shock and congestive heart failure. In response to a decrease in blood volume and low arterial pressure caused by these conditions, the plasma AVP level markedly increases, from a normal range of 1-10 pM to up to hundred-picomolar levels, to induce vasoconstriction. As this state is prolonged, the AVP concentration gradually declines below 30 pM within one hour. Further decreases in AVP levels lead to the failure of vasoconstriction, which may result in a low blood pressure that is insufficient for proper blood perfusion into organs, i.e., late-phase hemorrhagic shock. This is therapeutically addressed by administration of exogenous AVP to raise the arterial pressure. Thus, real-time measurement of plasma AVP concentrations is vital for timely and effective clinical intervention to prevent such life-threatening conditions.

Starting in the 1950s, antidiuretic bioassays were used to measure AVP at concentrations as low as 10 pM in rats by measuring the blood pressure response to intravenous injection of extracts in rats. The sensitivity was improved to measure AVP concentrations in plasma by introduction of additional exteriorized urinary bladders. The method has remained time consuming and to a large extent been replaced with immunoassays using AVP antibodies (RIA and ELISA). In the radio immunoassay (RIA), radiolabeled AVP is added and competes with the AVP in the sample in binding to AVP antibodies. The amount of radiolabel measured thus allows determination of the AVP concentration. In the competitive enzyme-linked immunoassay (ELISA), enzyme-linked AVP is applied and competes with the AVP in the sample to bind to surface-immobilized antibodies. The light signal induced by the subsequent enzymatic reaction is then used to quantify the AVP sample. As the current gold standard in AVP measurement, ELISAs eliminate the need for radioactive substances as required by RIAs, but are still cumbersome and require complicated protocols. In addition, as the reaction products can be easily immunosorbed on the plate during measurements, ELISAs are not amenable to real-time measurements.

Therefore a sensitive, specific, label-free, repeatable, and reversible nanosensor that can provide measurement of biomarkers in physiological fluids would represent a tremendous advance in the field.

### SUMMARY

This disclosure enables an aptamer-based graphene affinity nanosensor for detection of biomarkers, and a system and method for detecting biomarkers using an aptamer-based graphene affinity nanosensor. This disclosure at least partially addresses at least one of above inefficiencies. However, this disclosure can prove useful to other technical areas. Therefore, various claims recited below should not be construed as necessarily limited to addressing any of the above inefficiencies.

The present disclosure provides a device comprising a transistor comprising a graphene-based conducting channel, and a functional layer modified with an aptamer, and attached to the graphene based conducting channel via a polymer, wherein the aptamer binds with a target biomarker. In certain embodiments, the aptamer is an isolated enantiomer of D-ribose having SEQ ID NO:1. In other embodiments, the polymer is a polyethylene glycol. In yet other embodiments, a molecular weight of the PEG is from about 300 Da to about 3000 Da. In still other embodiments, the target biomarker is arginine vasopressin. In additional embodiments, a limit of detection of the device is from about 0.0001 pM to about 100 pM, where the limit of detection is a concentration of the target biomarker that is detectable by the device.

The present disclosure also provides a method for manufacturing an aptamer-based biosensor comprising creating a graphene-based conducting channel in a field effect transistor, coating the graphene-based conducting channel with a functional layer, wherein the functional layer is a polymer, and reacting an aptamer with the functional layer to form the aptamer-based biosensor. In some embodiments, the aptamer is an isolated enantiomer of D-ribose having SEQ ID NO:1. In other embodiments, the polymer is a polyethylene glycol. In yet other embodiments, a molecular weight of the PEG is from about 300 Da to about 3000 Da. In still other embodiments, the target biomarker is arginine vasopressin. In certain embodiments, a limit of detection of the device is from about 0.0001 pM to about 100 pM, where the limit of detection is a concentration of the target biomarker that is detectable by the device. In additional embodiments, the method further comprises reversing a reaction of the aptamer with the functional layer.

Additionally, the present disclosure provides a method for detecting a target biomarker comprising manufacturing a device comprising a transistor with a graphene-based conducting channel, coating the graphene-based conducting channel with a functional layer modified with an aptamer, measuring a first drain-source current of the device, exposing the device to a medium comprising a target biomarker, measuring a second drain-source current of the device, and determining binding of the aptamer to the target biomarker based on a difference between the first and second drain source currents. In various embodiments, the aptamer is an isolated enantiomer of D-ribose having SEQ ID NO:1. In certain embodiments, the polymer is a polyethylene glycol. In some embodiments, a molecular weight of the PEG is from about 300 Da to about 3000 Da. In some embodiments, the target biomarker is arginine vasopressin. In other embodiments, a limit of detection of the device is from 0.0001 pM to 100 pM, where the limit of detection is a concentration of the target biomarker that is detectable by the device. In additional embodiments, the method further comprises reversing a reaction of the aptamer with the functional layer.

It should be understood that the various individual aspects and features of this disclosure can be combined with any one or more individual aspect or feature, in any number, to form embodiments of the present invention that are specifically contemplated and encompassed by the present disclosure.

This disclosure is embodied in various forms illustrated in a set of accompanying illustrative drawings. Variations are contemplated as being a part of this disclosure, limited only by scope of various claims recited herein.

### BRIEF DESCRIPTION OF DRAWINGS

The set of accompanying illustrative drawings shows various exemplary embodiments of this disclosure. Such drawings are not to be construed as necessarily limiting this disclosure. Like numbers and/or similar numbering scheme can refer to like and/or similar elements throughout.

FIG. 1 is a representative illustration of an aptamer-based graphene affinity nanosensor according to an exemplary embodiment. The nanosensor is configured as a solution-gated graphene field-effect transistor.

FIG. 2 is a schematic of the surface of a graphene nanosensor functionalized with a biomarker-specific aptamer and polyethylene glycol (PEG) according to an exemplary embodiment.

FIG. 3 shows a scheme for graphene surface functionalization according to an exemplary embodiment. An aptamer is internally attached to a PEG molecule via an amino group.

FIG. 4A, FIG. 4B and FIG. 4C are illustrations of a secondary structure of the D-DNA AVP aptamer (FIG. 4A; SEQ ID NO:1), showing internal (FIG. 4B) and end (FIG. 4C) aptamer attachment strategies according to various exemplary embodiments.

FIG. 5A, FIG. 5B and FIG. 5C show characterization of a nanosensor. FIG. 5A is a photograph of a nanosensor for transfer curve characterization. FIG. 5B is a plot of a transfer curve characterization of a graphene nanosensor according to an exemplary embodiment before and after PASE, aptamer and ethanolamine modification. FIG. 5C is a micrograph of a nanosensor with source, drain electrodes and graphene channel.

FIG. 6 is Raman spectra of the graphene channel of a nanosensor according to an exemplary embodiment.

FIG. 7A and FIG. 7B are atomic force microscopy (AFM) images of bare graphene on a SiO₂/Si substrate (FIG. 7A) and graphene functionalized with aptamer (FIG. 7B).

FIG. 8A, FIG. 8B, FIG. 8C and FIG. 8D show the detection of AVP by a nanosensor in PBS buffer. Transfer characteristics of the aptamer-immobilized graphene measured when exposed to vasopressin solution, V_{Dirac} shifted to the negative gate voltage (FIG. 8A), Dirac point shift as a function of AVP concentration (FIG. 8B), reproducibility of a graphene nanosensor in AVP detection (FIG. 8C), and real-time measurement of AVP using a graphene nanosensor (AVP concentration range: 0 pM to 500 pM) (FIG. 8D).

FIG. 9A and FIG. 9B show results using graphene nanosensors. FIG. 9A shows responses of graphene nanosensors to analytes. FIG. 20A shows the specificity of a graphene nanosensor with aptamers internally attached. The Dirac point shift ΔV_{Dirac} showing the response of a nanosensor to different concentrations (1, 10, 100 and 1000 pM) of AVP and the control proteins (DAVP). FIG. 9B shows the effectiveness of internal-attachment AVP aptamer. The Dirac point shift ΔV_{Dirac} showing the response of a nanosensor to different concentrations (10, 100 and 1000 pM) of AVP using internal-attachment AVP aptamer and end-attachment AVP aptamer.

FIG. 10A and FIG. 10B are representative graphs showing the detection of AVP in ultrafiltrate with a PEG-modified nanosensor. The effect of PEG molecular weight (FIG. 10A) and real-time measurement (FIG. 10B) are shown.

### DETAILED DESCRIPTION

The present disclosure is now described more fully with reference to the set of accompanying illustrative drawings, in which exemplary embodiments of this disclosure are shown. This disclosure can be embodied in many different forms and should not be construed as necessarily being limited to the exemplary embodiments disclosed herein. Rather, the exemplary embodiments are provided so that this disclosure is thorough and complete, and fully conveys various concepts of this disclosure to those skilled in a relevant art.

The present disclosure provides devices to enable the sensitive and specific detection of biomarkers in microdialysis ultrafiltrates of physiological fluids, using an aptamer-based graphene affinity nanosensor. The nanosensor is a graphene field-effect transistor, in which a nucleic acid aptamer and a biomolecule-permeable polyethylene glycol (PEG) nanolayer are immobilized on the graphene surface. The aptamer is capable of specifically recognizing the target biomarker and induces a change in the carrier concentration of the graphene, which is measured to determine the biomarker concentration. The PEG nanolayer minimizes nonspecific adsorption of background molecules in the sample that would otherwise interfere with the biomarker detection.

The present disclosure provides an aptamer-based, graphene field-effect transistor (GFET) nanosensor capable of sensitive and specific detection of biomarkers in physiological fluids. For example, the present disclosure includes an approach for biomarker detection and measurement in microdialysis ultrafiltrates of physiological fluids, using an aptamer-based graphene affinity nanosensor. The nanosensor is a GFET in which the graphene conducting channel is protected or coated with a polyethylene glycol (PEG) nanolayer. The thickness of the PEG monolayer can vary and is selected to effectively suppress the adsorption of nonspecific molecules. The modification of graphene with PEG of varying molecular weights allows for minimization or suppression of binding of nonspecific molecules to the sensor, allowing for sensitive and specific detection of biomarkers in undiluted physiological fluids. With this ability to detect and measure biomarkers in undiluted physiological fluids, the nanosensor can be used in applications where sample dilutions are not practical.

The graphene conducting channel of this sensor is coated with a polyethylene glycol (PEG) nanolayer, with varying levels of thickness, to suppress the binding of nonspecific molecules to the sensor. 1-pyrenebutanoic acid succinimydyl ester (PASE) is anchored onto the graphene through π-π stacking interactions and for each PASE molecule, either an aptamer, for biomolecule detection, or PEG, for noise reduction, is covalently attached to PASE. Biomarker binding to the negatively charged aptamer redistributes the concentration of charged particles on the graphene surface resulting in different measured electrical currents relative to unbound aptamers and bare graphene. This allows for sensing human health states by measuring the presence and concentration of relevant biomarkers in physiological fluids.

In certain aspects of the present disclosure, the molecular weight of the PEG is from about 300 Da to about 3000 Da, about 350 Da to about 2600 Da, about 400 Da to about 2000 Da, about 450 Da to about 1500 Da, about 500 Da to about 1000 Da, and any other suitable molecular weights between these ranges. In other embodiments, that weight of PEG is about 300 Da, about 325 Da, about 350 Da, about 375 DA, about 400 Da, about 425 Da, about 450 Da, about 475 Da, about 500 Da, about 525 Da, about 550 Da, about 575 Da, about 600 Da, about 625 Da, about 650 Da, about 675 Da, about 700 Da, about 725 Da, about 750 Da, about 775 Da, about 800 Da, about 825 Da, about 850 Da, about 875 Da, about 900 Da, about 925 Da, about 950 Da, about 975 Da, about 1000 Da, about 1025 Da, about 1050 Da, about 1075 Da, about 1100 Da, about 1125 Da, about 1150 Da, about 1175 Da, about 1200 Da, about 1225 Da, about 1250 Da, about 1275 Da, about 1300 Da, about 1325 Da, about 1350 Da, about 1375 DA, about 1400 Da, about 1425 Da, about 1450 Da, about 1475 Da, about 1500 Da, about 1525 Da, about 1550 Da, about 1575 Da, about 1600 Da, about 1625 Da, about 1650 Da, about 1675 Da, about 1700 Da, about 1725 Da, about 1750 Da, about 1775 Da, about 1800 Da, about 1825 Da, about 1850 Da, about 1875 Da, about 1900 Da, about 1925 Da, about 1950 Da, about 1975 Da, about 2000 Da, about 2025 Da, about 2050 Da, about 2075 Da, about 2100 Da, about 2125 Da, about 2150 Da, about 2175 Da, about 2200 Da, about 2225 Da, about 2250 Da, about 2275 Da, about 2300 Da, about 2325 Da, about 2350 Da, about 2375 DA, about 2400 Da, about 2425 Da, about 2450 Da, about 2475 Da, about 2500 Da, about 2525 Da, about 2550 Da, about 2575 Da, about 2600 Da, about 2625 Da, about 2650 Da, about 2675 Da, about 2700 Da, about 2725 Da, about 2750 Da, about 2775 Da, about 2800 Da, about 2825 Da, about 2850 Da, about 2875 Da, about 2900 Da, about 2925 Da, about 2950 Da, about 2975 Da, about 3000 Da, and any other suitable molecular weights between these ranges.

In various embodiments of the present disclosure, the limit of detection (LOD) of the device is from 0.0001 pM to 100 pM, where the limit of detection is a concentration of the target biomarker that is detectable by the device. The LOD can be from about 0.001 pM to about 100 pM, about 0.01 pM to about 90 pM, about 0.05 pM to about 80 pM, about 0.1 pM to about 70 pM, about 0.5 pM to about 60 pM, about 1 pM to about 55 pM, about 1.5 pM to about 50 pM, about 5 pM to about 40 pM, and any other suitable LOD within these ranges. In certain embodiments, the limit of detection (LOD) of the device is about 0.0001 pM, about 0.0005 pM, about 0.0010 pM, about 0.0015 pM, about 0.0020 pM, about 0.0025 pM, about 0.0030 pM, about 0.0035 pM, about 0.0040 pM, about 0.0045 pM, about 0.0050 pM, about 0.0055 pM, about 0.0060 pM, about 0.0065 pM, about 0.0070 pM, about 0.0075 pM, about 0.0080 pM, about 0.0085 pM, about 0.0090 pM, about 0.0095 pM, about 0.0100 pM, about 0.015 pM, about 0.020 pM, about 0.025 pM, about 0.030 pM, about 0.035 pM, about 0.040 pM, about 0.045 pM, about 0.050 pM, about 0.055 pM, about 0.060 pM, about 0.065 pM, about 0.070 pM, about 0.075 pM, about 0.080 pM, about 0.085 pM, about 0.090 pM, about 0.095 pM, about 0.100 pM, about 0.110 pM, about 0.120 pM, about 0.130 pM, about 0.140 pM, about 0.150 pM, about 0.160 pM, about 0.170 pM, about 0.180 pM, about 0.190 pM, about 0.20 pM, about 0.30 pM, about 0.40 pM, about 0.50 pM, about 0.60 pM, about 0.70 pM, about 0.80 pM, about 0.90 pM, about 1 pM, about 2 pM, about 3 pM, about 4 pM, about 5 pM, about 6 pM, about 7 pM, about 8 pM, about 9 pM, about 10 pM, about 11 pM, about 12 pM, about 13 pM, about 14 pM, about 15 pM, about 16 pM, about 17 pM, about 18 pM, about 19 pM, about 20 pM, about 21, pM, about 22 pM, about 23 pM, about 24 pM, about 25 pM, about 26 pM, about 27 pM, about 28 pM, about 29 pM, about 30 pM, about 31 pM, about 32 pM, about 33 pM, about 34 pM, about 35 pM, about 36 pM, about 37 pM, about 38 pM, about 39 pM, about 40 pM, about 41, pM, about 42 pM, about 43 pM, about 44 pM, about 45 pM, about 46 pM, about 47 pM, about 48 pM, about 49 pM, about 50 pM, about 51 pM, about 52 pM, about 53 pM, about 54 pM, about 55 pM, about 56 pM, about 57 pM, about 58 pM, about 59 pM, about 60 pM, about 61, pM, about 62 pM, about 63 pM, about 64 pM, about 65 pM, about 66 pM, about 67 pM, about 68 pM, about 69 pM, about 70 pM, about 71 pM, about 72 pM, about 73 pM, about 74 pM, about 75 pM, about 76 pM, about 77 pM, about 78 pM, about 79 pM, about 80 pM, about 81, pM, about 82 pM, about 83 pM, about 84 pM, about 85 pM, about 86 pM, about 87 pM, about 88 pM, about 89 pM, about 90 pM, about 91 pM, about 92 pM, about 93 pM, about 94 pM, about 95 pM, about 96 pM, about 97 pM, about 98 pM, about 99 pM, about 100 pM, and any other suitable LOD within these ranges.

An aptamer is capable of specifically recognize the target biomarker and induce a change in the carrier concentration of the graphene, which is measured to determine the biomarker concentration. The PEG nanolayer minimizes nonspecific adsorption of background molecules in the sample that would otherwise interfere with the biomarker detection. The graphene nanosensors of the present disclosure are functionalized with a new aptamer, which for the first time enables real-time measurement of arginine vasopressin (AVP). Arginine vasopressin (AVP), an anti-diuretic hormone, is a clinically important low-molecular-weight biomarker (1084 Da) as well as a therapeutic molecule for severe hemorrhage, septic shock and congestive heart failure. In response to a decrease in blood volume and low arterial pressure caused by these conditions, the plasma AVP level markedly increases, from a normal range of 1 pM to 10 pM to up to hundred-picomolar levels, to induce vasoconstriction. As this state is prolonged, the AVP concentration gradually declines below 30 pM within one hour. Further decreases in AVP levels lead to the failure of vasoconstriction, which may result in a low blood pressure that is insufficient for proper blood perfusion into organs, i.e., late-phase hemorrhagic shock. This is therapeutically addressed by administration of exogenous AVP to raise the arterial pressure. Thus, real-time measurement of plasma AVP concentrations is vital for timely and effective clinical intervention to prevent such life-threatening conditions.

The aptamer is based on the natural enantiomer D-ribose and offers high sensitivity (sub-pMs) and rapid responsiveness (several minutes) to AVP concentration changes, thereby enabling real-time monitoring. Also, unlike existing aptamer-based graphene sensors that use end-attached aptamers, i.e., aptamers attached at the 5′ end to graphene, the aptamer described in the present disclosure is attached to the graphene surface at an internal position. This internal aptamer attachment strategy effectively brings the molecular complex formed due to the aptamer-AVP binding into closer proximity to the graphene surface, thereby generating a strong sensor response to low concentrations of AVP.

In addition, the graphene surface is passivated with a nanolayer of polyethylene glycol (PEG) to minimize nonspecific adsorption of background molecules to the graphene surface, enabling specific measurement of AVP in physiological fluids such as dialysis ultrafiltrates.

The following example is included to demonstrate illustrative embodiments of the present disclosure. It should be appreciated by those of skill in the art that the techniques disclosed in the examples that follow represent techniques discovered by the inventors to function well in the practice of the present disclosure, and thus can be considered to constitute one embodiment of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Real-Time Label-Free Measurement of Vasopressin by a Graphene Affinity Nanosensor with an Internally Surface-Attached Aptamer

This example presents an aptameric graphene nanosensor for highly sensitive measurement of arginine vasopressin (AVP). The nanosensor is a field-effect transistor (FET) with monolayer graphene as the conducting channel. A long-chain AVP-specific aptamer that is internally attached to the graphene is used for AVP recognition. The negatively charged aptamer folds into a closed T-junction conformation upon binding with AVP in the immediate vicinity of the graphene. This sensitively changes the carrier density of the graphene and induces a change in the electrical characteristics of the FET, which can be measured to determine the AVP concentration. Results have shown that the nanosensor is capable of quantitative measurement of AVP in a concentration range of 0.3 pM to 1 nM in phosphate-buffered saline (PBS) and microdialysis ultrafiltrates. The nanosensor has applications in real-time monitoring of plasma AVP concentrations in clinical care of patients with life threatening conditions such as severe hemorrhage, septic shock and congestive heart failure.

Studies have been reported on improved AVP detection methods. Carbon nanotubes were used to construct an electrochemical sensor for detection of AVP with a limit of detection (LOD) of 43 pM phosphate buffered saline (PBS) buffer. An optofluidic surface enhanced Raman spectroscopy (SERS) device was reported for on-chip detection of AVP with an LOD of 11.3 pM in PBS. The inventors previously reported aptamer-based enrichment and detection of AVP by methods including nanostructured SERS, matrix-assisted laser desorption/ionization mass spectrometry (MALDI-MS), achieving LODs down to 1 pM in conditioned media as well as 10 pM in microdialysis ultrafiltrates. These approaches, however, are still not sufficiently sensitive for AVP concentration ranges that must be measured in clinical applications (<10 pM). In addition, the AVP detection studies by the inventors and others used spiegelmers, which are aptamers with mirror image monomeric units (based on L-ribose). These L-DNA (with low affinity to AVP) or L-RNA aptamers (with ultra-high affinity), in addition to requiring expensive and time-consuming synthesis procedures, have small dissociation rate constants. As a result, when binding to AVP, they form complexes that have hours-long dissociation half-times, which preclude real-time monitoring of AVP concentration changes.

This example presents an approach to real-time label-free AVP measurements in physiological fluids using an aptamer-based graphene nanosensor. As a two-dimensional carbon-based nanomaterial with large surface-to-volume ratio and high electrical carrier mobility, graphene has been actively used in biological and chemical sensing for detection of analytes such as ions, proteins, and nucleic acids. Here, graphene nanosensors functionalized with a new aptamer are explored for the first time to enable real-time measurement of AVP. The new D-DNA AVP aptamer is based on the natural enantiomer D-ribose and has been developed to offer a highly sensitive (sub-pMs) and rapid responsiveness (several minutes) to AVP concentration changes, thereby enabling real-time monitoring. Also, unlike existing aptamer-based graphene sensors that use end-attached aptamers, i.e., aptamers attached at the 5′ end to graphene, the presently described aptamer is attached to the graphene surface at an internal position. This internal aptamer attachment strategy effectively brings the molecular complex formed due to the aptamer-AVP binding into closer proximity to the graphene surface, thereby generating a strong sensor response to low concentrations of AVP. In addition, the graphene surface is passivated with a nanolayer of polyethylene glycol (PEG) to minimize nonspecific adsorption of background molecules to the graphene surface, enabling specific measurement of AVP in physiological fluids such as dialysis ultrafiltrates. As a result, the nanosensor allows direct and label-free AVP measurement of AVP at clinically appropriate concentrations in a real-time manner, which enables timely identification of abnormal AVP concentrations in patients for improved clinical outcome. This is in contrast to existing graphene-based aptameric affinity sensors, which often show a lack of reversibility in their responses to biomarkers and hence preclude real-time monitoring. The present results show that the device is capable of repeatable specific and reversible detection of AVP in PBS buffer as well as human plasma ultrafiltrates with detection limits down to 0.3 pM.

### Aptamer Design

To overcome the low dissociation rate constants of spiegelmers previously used for AVP detection, a new anti-AVP aptamer was developed using the natural enantiomer D-ribose via a solution-phase isolation method. This long-chain 66-nt D-DNA aptamer (sequence:5′-CTCTCGGGACGACGTCCAAGTAGTACGTTTAATTAGGATTTCCGAATTATTGGCATG CGTCGTCCC-3′; SEQ ID NO:1) forms a stem-loop structure (FIG. 4A). To be internally attached to graphene, the aptamer was modified at the desired internal attachment position with an amino linker (FIG. 4A and FIG. 4B). For end-attachment to graphene, the aptamer was modified at the 5′ end (FIG. 4C). When binding to AVP, the aptamer undergoes large conformational changes close to its stem, with concomitant rearrangements of accompanying ion clouds, so as to change the electrical conductivity of the graphene and produce a measurable signal in the FET.

### Nanosensor Design and Preparation

### Design

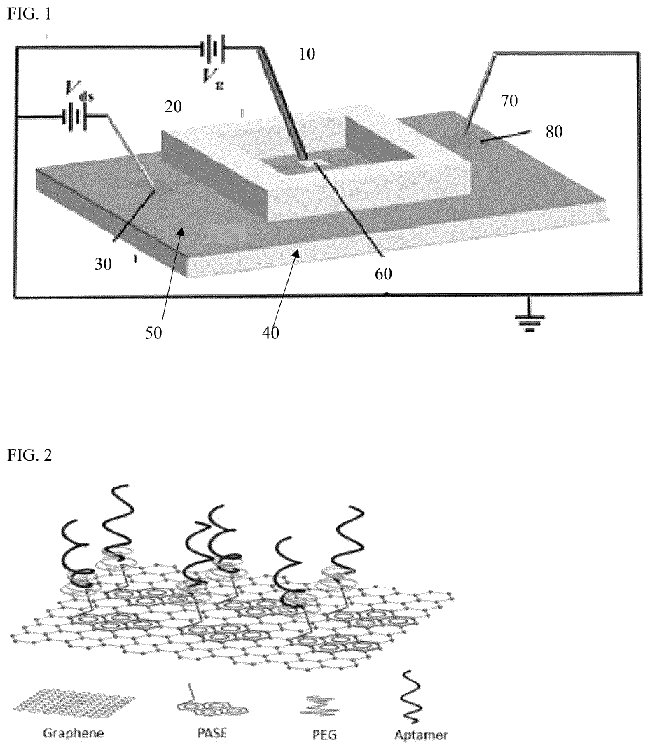

The nanosensor is an electrolyte-gated graphene FET, in which chemical vapor deposition (CVD)-prepared graphene was patterned between the source and drain electrodes as the conduction channel. A standard Ag/Cl reference electrode was inserted into the solution as the gate during AVP measurements (FIG. 1). As shown in FIG. 1, the nanosensor includes an electrode 10, a PDMS well 20, a drain 30, a silicon wafer 40 with an silica surface 50, a graphene channel 60, a probe 70 and a source 80. Each measurement was repeated three times in the experiments.

### Fabrication

To fabricate the nanosensor, Au/Ti electrodes as the drain and source were subsequently fabricated using E-beam evaporation and lift-off on 300 nm SiO₂/Si substrate. Chemical vapor deposition (CVD) synthesized graphene was transferred onto the electrodes and then patterned to define the conducting channel using photolithography and oxygen plasma etching. The polymethyl methacrylate (PMMA) carrier on graphene was removed by acetone at last.

### Functionalization

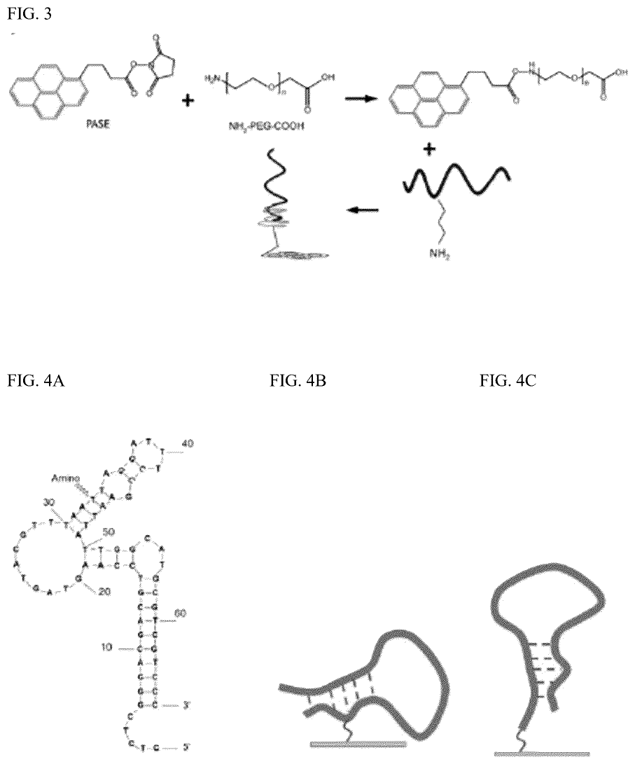

To functionalize the nanosensor after fabrication (FIG. 2 and FIG. 3): First, the graphene-based device was immersed in 10 mM PASE solution for 5 hours at room temperature, sequentially rinsed with dimethylformamide (DMF) and PBS buffer to remove free PASE. Second, the graphene nanosensor was rinsed with PBS followed by incubating in 100 nM internal AVP aptamer solution overnight at room temperature. After rinsing with PBS, 100 mM ethanolamine was added onto the graphene channel for 1 hour to deactivate and block the excess reactive groups remaining on graphene surface. To enable and evaluate AVP detection in ultrafiltrate, 1000 Da PEG was immobilized on PASE by immersing the nanosensor in 2 mM PEG solution for 10 hours, followed by aptamer decoration and ethanolamine passivation (FIG. 3).

### Materials

Long-chain D-DNA AVP aptamers for either internal or end attachment scheme were synthesized and purified by Integrated DNA Technologies (Coralville, IA). AVP and the control molecule desmopressin (DAVP) were purchased from American Peptide Company Inc. (Sunnyvale, CA). The ultrafiltrate sample was prepared by filtering the human serum using a Vivaspin centrifugal concentrator with a molecular weight cut-off (MWCO) of 20 kDa. Chemical vapor deposition (CVD) graphene grown on annealed copper (Cu) foil (99.9%, 25 m thick) was purchased from Mingri Cemented Carbide (Zhuzhou, China). Chemicals and reagents for surface functionalization: Polydimethylsiloxane (PDMS, Sylgard-184) was purchased from Dow Corning (Midland, MI) for PDMS chamber. NH₂—(CH₂CH₂O)ₙ—COOH (PEG, also known as serial PEG), 1-pyrenebutanoic acid succinimidyl ester (PASE) and PBS were purchased from Sigma-Aldrich (St. Louis, MO).

### Device Characterization

### Transfer Curve Characterization

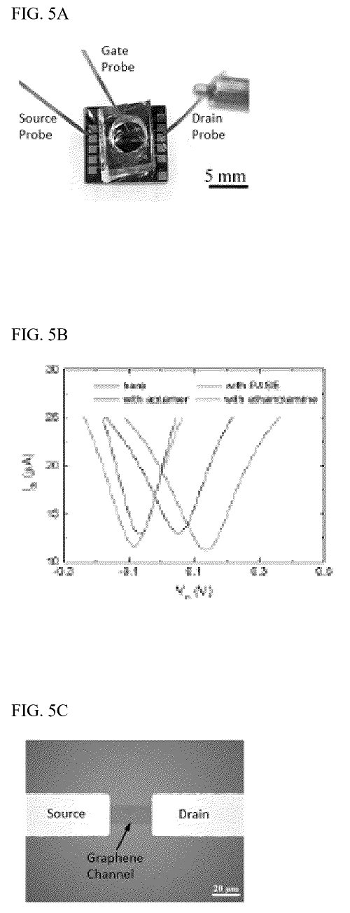

In measurements, a PDMS well is mounted on the substrate of the nanosensor to hold the PBS solution (FIG. 5A). The measured transfer characteristic curves, i.e., plots of the drain-source current (I_{ds}) vs. the gate voltage (Vg) at a given drain-source voltage (V_{ds}=4 mV) (FIG. 5B) were used to verify the functionalization of the nanosensor. The Dirac point (V_{Dirac}), the value of V_{g} at which I_{ds} reaches its minimum, was observed to increase from 61 to 132 mV after PASE immobilization, indicating PASE-induced p-type doping in the graphene. Upon attachment of the aptamer, which induced n-type doping in the graphene, V_{Dirac} then decreased to −82 mV. After modification of the graphene by ethanolamine, V_{Dirac} shifted from −82 to approximately −90 mV due to the induced n-type doping.

### Raman Spectra

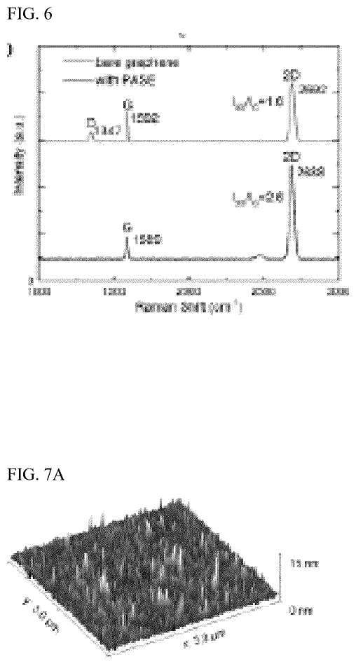

Graphene was patterned between the source and drain electrodes as the conduction channel (FIG. 5C). Raman spectra of the graphene channel before and after PASE immobilization were observed (FIG. 6). The split of G-band after PASE modification demonstrated the successful coupling of graphene and the pyrene groups on PASE via π-π interaction.

### AFM

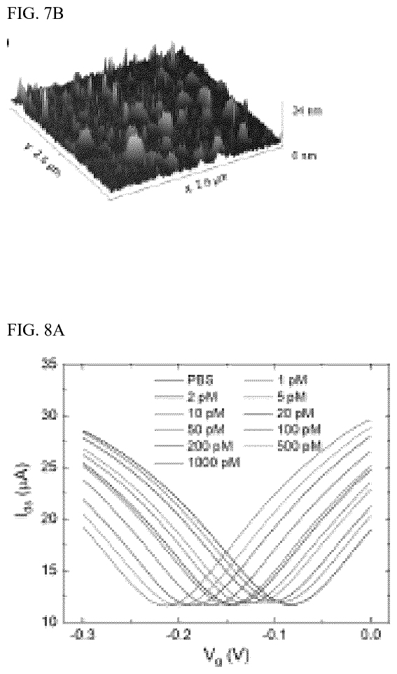

The modification of graphene was verified using AFM and transfer characteristic curves (FIG. 7A and FIG. 7B). The roughness (Ra) of bare graphene is about 3 nm which is determined by the SiO₂/Si substrate while the peaks can reach a height of 12 nm (FIG. 7A). After functionalization process, AVP aptamer was successfully immobilized on the graphene surface (FIG. 7B) via PASE linker as Ra was increased to 6 nm with larger peaks (20 nm).

### Measurements in Controlled Media

### AVP Measurement in PBS

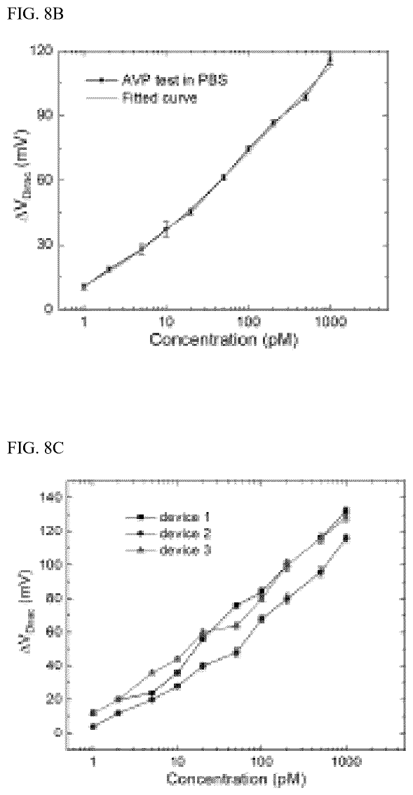

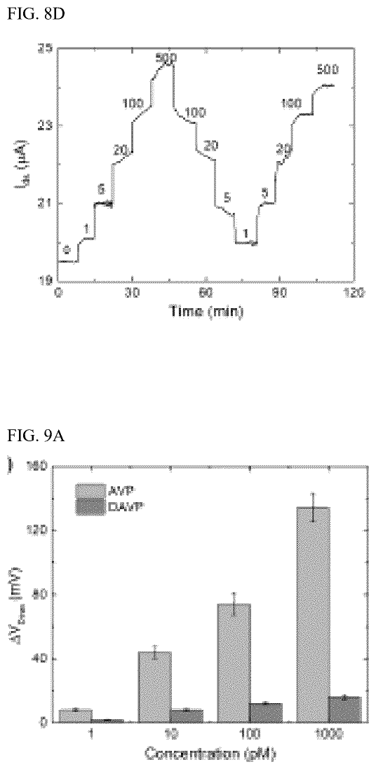

The nanosensor was first tested with measurements of AVP in PBS. The gate voltage V_{g} was varied from −300 to 0 mV while the drain-source voltage V_{ds} was fixed at 4 mV during the measurements (FIG. 8A). AVP solution was filled in a PDMS well mounted on the device chip. As the AVP concentration, denoted by c, increased from 1 pM to 1 nM, the Dirac point V_{Dirac} decreased (i.e., shifted to the left) consistently from −84 to −208 mV (FIG. 8A), reflecting increasing numbers of AVP molecules being captured by the surface-bound aptamer. The aptamer-captured AVP was brought to the close vicinity of the graphene surface as the aptamer switched from a chain structure to a stable, closed T-junction conformation. With an isoelectric point (pI) of 10.9, these AVP molecules were positively charged in PBS (pH 7.4), and hence induced electrons (i.e., n-type dopants) in the graphene, thereby causing the observed left shift of the Dirac point.

### Repeated Measurements

The nanosensor was then tested with repeated AVP measurements. For a given nanosensor, the shift of the Dirac point, ΔV_{Dirac}=V_{Dirac}−V_{Dirac,0} where V_{Dirac,0} is the Dirac point in AVP-free PBS buffer, was measured in three repeats, each with a series of AVP concentrations.

### Repeatability

Between the repeat experiments, the device was regenerated by washing with PBS buffer. The measurement results (FIG. 8B) showed that the device response was repeatable, with highly consistent values of ΔV_{Dirac} (standard errors <5%) at each AVP concentration tested.

### Detection Limit

The responsivity of the device (i.e., the rate of Dirac point shift to the change of AVP concentration) was determined to be approximately R=9.7 mV/pM at low concentrations of AVP (approaching 1 pM). This responsivity, along with the standard error of the Dirac point measurements (s=0.9 mV), allowed the limit of detection (LOD) to be estimated: LOD=3s/R=0.3 pM.

### Reproducibility

Various nanosensors were also used to evaluate the reproducibility of the nanosensor (FIG. 8C). The response showed a consistency with deviations less than 20% at any given AVP concentration. Such variation of the measurement results for each nanosensor may be caused by the quality of graphene transfer onto different sensor chips as well as the following functionalization process. The consistency of the nanosensors can be improved by normalization, i.e., Δy=ΔV_{Dirac}/ΔV_{Dirac,max} where ΔV_{Dirac,max} is the Dirac point shift at 1000 pM AVP concentration.

### Real-Time Monitoring of AVP

To assess the capability of the nanosensor to allow real-time monitoring of AVP, its response (the drain-source current I_{ds} with the gate voltage fixed at −20 mV) was measured at time-dependent AVP concentrations, [AVP](FIG. 8D). [AVP] was initially at 0 pM with a baseline device response of I_{ds}=19.5 μA. Starting at time t=7.5 min, [AVP] was increased consecutively to 1, 5, 20 and 100 pM (reaching 500 pM at t=38 min), then decreased through these values in descending order (reaching 1 pM at t=72 min), and finally increased through these values again (reaching 500 pM at t=104 min). In this time course, each of the two extreme [AVP] values (1 and 500 pM) was attained once in an ascending manner (i.e., the value was reached “from below” in a [AVP]-increasing process), and once in a descending manner (i.e., the value was reached “from above” in an [AVP]-decreasing process). Each of the intermediate test [AVP] values (5, 20 and 100 pM) was attained twice ascendingly and once descendingly. The resulting measurements of I_{ds} agreed within 1.7% at each of these test [AVP] values, regardless of whether the AVP concentration was reached ascendingly or descendingly. For example, for [AVP]=5 pM, I_{ds} respectively achieved steady values of 21.0, 20.8, and 20.9 μA (agreeing within 0.5%) over the time intervals [15.1, 22.0] min ([AVP] increasing), [64.1, 72.2] min ([AVP] decreasing) and [81.0, 88.1] min ([AVP] increasing again). This indicates that the nanosensor response was repeatable and reversible, satisfying a critical requirement of real-time AVP monitoring.

For each of these AVP concentration changes, a steady value of the device response I_{ds} was achieved with a 63%-time constant of ˜2 min. This time response, which is considered appropriate for real-time monitoring of AVP in clinical settings, was primarily determined by the time for AVP molecules to diffuse and equilibrate near the sensor surface, which could be further improved by optimizing the measurement protocols (e.g., using flow-through mode) to allow faster aptamer-AVP reaction.

### Specificity

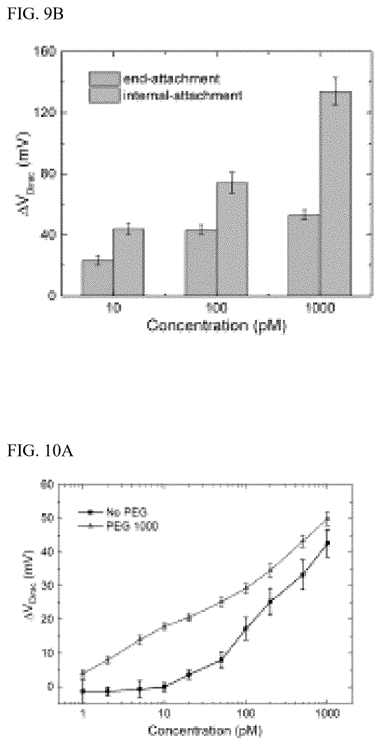

To examine the specificity of the nanosensor in AVP detection, desmopressin (DAVP), a synthetic analog of AVP, was used as the control. The nanosensor was exposed to the control protein at different concentrations (1, 10, 100 and 1000 pM) in PBS (FIG. 9A) which was consistent with AVP detection. The Dirac point shift ΔV_{Dirac} for AVP at 1 pM was four times larger than that for control proteins at the same concentration and the difference was larger at higher concentration (i.e., ΔV_{Dirac} for AVP at 100 pM was seven times larger than that for the control protein), which shows the aptamer can bind to AVP specifically.

### Effect of Aptamer Attachment Position

To examine the effect of internally attaching the aptamer to the graphene, the nanosensors were compared to those with end aptamer attachment in AVP measurement (FIG. 9B). Both types of nanosensors were exposed to AVP in PBS at different concentrations (1, 10, 100, 1000 pM). The Dirac point shift ΔV_{Dirac} using internal attachment aptamer was on average two times larger than that using end attachment aptamer. The standard error was comparable to the mean of ΔV_{Dirac} at 1 pM using end attachment aptamer, suggesting an LOD of ˜1 pM. With the internally attached aptamer, the LOD was estimated to be 0.3 pM assuming the same level of standard error when AVP was diluted to concentrations lower than 1 pM. The results show that the molecular complex formed by the aptamer-AVP binding in the closer proximity of the surface induced more significant electrostatic gating effect in the graphene channel, thus leading to improved measurement results.

### Measurements in Microdialysis Ultrafiltrates

To allow the nanosensor to operate reliably in physiological media, e.g., microdialysis ultrafiltrates, the nanosensor was functionalized with a nanolayer of PEG. The ability of PEG to reduce nonspecific adsorption on the device was tested by comparing the PEG-functionalized nanosensors to those not coated with PEG for AVP measurements in ultrafiltrates.

### Without PEG

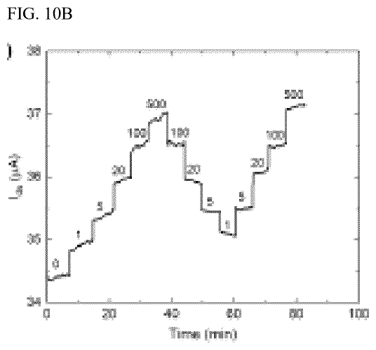

The nanosensor, without modification with PEG, was tested in microdialysis ultrafiltrates (pH=7.4). There was no measurable response at the lowest AVP concentrations tested (1 through 10 pM). The response was measured at higher AVP concentrations, with the Dirac point shift ΔV_{Dirac} increasing from 4±1 mV at 20 pM to 48±5 mV at 1000 pM (FIG. 10A). The LOD was estimated to be 15 pM, almost two orders of magnitude higher than that in PBS (0.3 pM). In addition, the noise level, i.e, the maximum standard error (normalized with the mean) of Dirac point shift measurements, increased from 5% to 17% as the medium was changed from PBS to ultrafiltrates. This significant decrease in performance was consistent with the effects of microdialysis ultrafiltrates contained a large variety of interferents, such as small molecules as well as proteins, hormones, microorganisms which have sufficiently low molecular weights to permeate through the microdialysis membrane and adsorb onto the graphene surface.

### With PEG

Nanosensors that were coated with a PEG nanolayer (1000 Da) were then used to measure AVP in ultrafiltrates (FIG. 10A). A consistent left shift of the Dirac point was observed over the entire AVP concentration range, including concentrations below 10 pM that were not measurable when the devices were not protected by PEG. The sensor's LOD was estimated to be 0.4 pM, both of which were comparable to the measurement results in PBS. In addition, with PEG preventing charged interferents from adsorbing to the graphene surface, the PEG-coated nanosensors offered a significantly improved repeatability in AVP measurements in ultrafiltrates, with the coefficient of standard errors at the measurement points decreasing to 7% from 16% in the absence of a PEG coating.

### Real-Time

Towards real-time monitoring of AVP, the device response (the drain-source current I_{ds} with the gate voltage fixed at −20 mV) was also measured at time-dependent AVP concentrations in ultrafiltrates (FIG. 10B). Similar to the time-dependent testing in PBS buffer (FIG. 8D) described above, starting at time t=7.2 min, [AVP] was increased (from 0 pM) consecutively to 1, 5, 20, 100 and 500 pM, then decreased to 1 pM, and finally increased again to 500 pM. Similar to the results from testing in PBS, the measured I_{ds} agreed within 0.6% at each of these test [AVP] values, regardless of whether the AVP concentration was reached ascendingly or descendingly. For example, for [AVP]=5 pM, I_{ds} respectively achieved steady values of 35.3, 35.4, and 35.5 μA (agreeing within 0.3%) over the time intervals [14.7, 21.2] min ([AVP] increasing), [49.6, 55.5] min ([AVP] decreasing) and [60.6, 65.8] min ([AVP] increasing again). This again indicates that the nanosensor response was repeatable and reversible, satisfying a critical requirement of real-time AVP monitoring. Also, for each of these AVP concentration changes, a steady value of the device response I_{ds} was achieved with a 63%-time constant of ˜2 min, which is appropriate for real-time monitoring of AVP in clinical settings.

### Nanosensor Summary

Finally, the performance of the nanosensor for AVP detection was compared to several other reported AVP detection methods in Table 1, which shows that the presently described AVP nanosensor offers the lowest LOD in both conditioned and physiological media and is also the only device that has achieved real-time monitoring of AVP.

TABLE 1
Comparison of response characteristics of the
nanosensor and other AVP detection methods

| | | | | LOD in | | |
|---|---|---|---|---|---|---|
| | | LOD in | | Physiological | | Real-time |
| Detection Method | Aptamer | Buffer | | Media | | Detection |
| CNT | L-DNA | 43 | pM | — | | No |
| SERS | NOX-F37 | 11.3 | pM | — | | No |
| Nanostructured | NOX-F37 | 1 | pM | — | | No |
| SERS | | | | | | |
| MALDI-MS | NOX-F37 | 1-10 | pM | — | | No |
| MALDI-TOF-MS | NOX-F37 | 1 | pM | 10 | pM | No |
| Present | D-DNA | 0.3 | pM | 0.5 | pM | Yes |

| | |
|---|---|
| Nanosensor | Aptamer |

### Full Reversibility

The results above showed that the graphene FET nanosensor can sensitively detect AVP at physiologically relevant concentrations. Of particular significance is the device's fully reversible response to AVP. That is, unlike existing affinity biosensors that are often affected by irreversible binding between receptors (antibodies or aptamers) and ligands at room temperature, the nanosensor was highly reversible in the entire concentration ranges used. Unlike other aptamer-ligand interactions for which dissociation requires the use of elevated temperatures or reaction-promoting ions, the dissociation of bound AVP molecules from the aptamer occurred spontaneously with a time constant about 2 minutes upon a decrease of AVP concentration in solution. This reversibility makes the nanosensor reusable and applicable to analyte concentrations that may both increase and decrease in the course of measurements, as is the case for real-time monitoring of AVP in clinical settings.

### Mechanism

Without being bound by any one specific theory, the reversibility of the device could be explained by the kinetics of binding of AVP with the aptamer. When there was a decrease in the AVP concentration, the non-covalent hydrogen bonds within AVP-aptamer complexes that previously formed on the graphene surface might be easily disrupted (resulting in breakage of the AVP-aptamer complex and regeneration of the aptamer) until the binding between AVP and aptamer molecules reached a new equilibrium. Thus, the nanosensor is highly repeatable and reversible in AVP measurement.

Internal Attachment Vs. End Attachment

The signal levels of aptameric graphene sensors can be significantly impacted by the distance between AVP-aptamer complexes and the graphene surface. Hence, a surface attachment scheme was designed such that the aptamer was attached internally to the graphene. Compared to the commonly used end aptamer attachment scheme (i.e., the aptamer is attached to the surface at the 5′ end), the internal attachment strategy was expected to bring the AVP-aptamer complex closer to the graphene surface. Indeed, measurements in PBS with the aptamer internally attached showed a total shift of the Dirac point by 128 mV over an AVP concentration range of 0 to 1000 pM, as compared to the 58 mV Dirac point shift over the same AVP concentration range when the aptamer was end-attached to the device (FIG. 9B). Correspondingly, the internal aptamer attachment scheme achieved an LOD of 0.3 pM, which was also significantly lower than that allowed by the end aptamer attachment method (1 pM).

### Comparing Measurements in Ultrafiltrates and in PBS

The nanosensor was capable of detecting AVP in a full range of clinically relevant (sub-pM to ˜100 pM) concentrations in both conditioned (PBS) buffer and physiological fluids (ultrafiltrates). With an LOD down to the sub-picomolar level, the device achieved a high, clinically appropriate sensitivity that had not been demonstrated with any other methods for clinical AVP measurements. The following insights were learned by comparing measurements in ultrafiltrates and in PBS, with and without the device protected by PEG.

### Nonspecific Adsorption Deteriorating Measurement Resolution

First, when not coated with PEG, the nanosensor incurred considerably higher measurement noise and lower response signal levels in ultrafiltrates. AVP was quantitatively measured down to 1 pM in PBS buffer (with an estimated LOD=0.3 pM), but only at concentrations higher than 10 pM in ultrafiltrates (LOD=15 pM) (FIG. 9B and FIG. 10A). This deterioration in the sensor performance was attributable to nonspecific adsorption of charged molecules such as permeable proteins, hormones to the graphene surface that introduced non-specific signal as well as occluded binding sites.

### PEG Allowing AVP Detection in Ultrafiltrates

Second, protecting the nanosensor with PEG allowed sensitive detection of AVP in ultrafiltrates. When coated with PEG of molecular weight 1000 Da, the device was capable of quantitatively measuring AVP at the full concentration range tested (1-1000 pM), with an estimated LOD=0.4 pM. The PEG protection led to significantly reduced noise levels (e.g., from 3.5 mV in the absence of PEG to 1.0 mV with PEG coating when the AVP concentration was 1 pM) and improved signal-to-noise ratios (SNRs) in ultrafiltrates (e.g., from signal indistinguishable from noise to 4:1 at 1 pM). As such, the PEG-protected sensor achieved the quantitative detection of AVP at low pM-level concentrations. This improvement is important for clinical applications and was affected by the PEG polymer forming a permeable layer on the graphene surface to reduce the nonspecific binding preventing interferents in ultrafiltrates from adsorbing to the graphene surface.

In summary, a graphene-based nanosensor was developed for highly sensitive, specific and label-free AVP detection in physiological medium. The decorated aptamer specifically binds with the AVP and induced a change in carrier concentration of the graphene through electrostatic gating effect, which was measured to determine the biomarker concentration. The AVP detection was demonstrated to be highly repeatable, reproducible, and can be performed in a real-time manner. The immobilized biomolecule-permeable PEG layer enabled the nanosensor for AVP detection down to 1 pM with a theoretic limit of detection at 0.5 pM. As a result, the nanosensor offers a high degree of sensitivity and specificity for AVP measurement in physiological solutions, and can hence be used for real-time monitoring of AVP conditions in the human body.

Features described with respect to certain exemplary embodiments can be combined and sub-combined in and/or with features described with respect to various other exemplary embodiments, even if such combinations and/or sub-combinations are not specifically described herein. Also, different aspects and/or elements of exemplary embodiments, as disclosed herein, can be combined and sub-combined in a similar manner as well. Further, some exemplary embodiments, whether individually and/or collectively, can be components of a larger system, wherein other procedures can take precedence over and/or otherwise modify their application. Additionally, a number of steps can be required before, after, and/or concurrently with exemplary embodiments, as disclosed herein. Note that any and/or all methods and/or processes, at least as disclosed herein, can be at least partially performed via at least one entity in any manner.

Various terminology used herein can imply direct or indirect, full or partial, temporary or permanent, action or inaction. For example, when an element is referred to as being “on,” “connected” or “coupled” to another element, then the element can be directly on, connected or coupled to the other element and/or intervening elements can be present, including indirect and/or direct variants. In contrast, when an element is referred to as being “directly connected” or “directly coupled” to another element, there are no intervening elements present.

Although the terms first, second, etc. can be used herein to describe various elements, components, regions, layers and/or sections, these elements, components, regions, layers and/or sections should not necessarily be limited by such terms. These terms are used to distinguish one element, component, region, layer or section from another element, component, region, layer or section. Thus, a first element, component, region, layer, or section discussed herein could be termed a second element, component, region, layer, or section without departing from various teachings of this disclosure.

Various terminology used herein is for describing particular exemplary embodiments and is not intended to be necessarily limiting of this disclosure. As used herein, various singular forms “a,” “an” and “the” are intended to include various plural forms as well, unless a context clearly indicates otherwise. Various terms “comprises,” “includes,” “comprising” and/or “including” when used in this specification specify a presence of stated features, integers, steps, operations, elements, and/or components, but do not preclude the presence and/or addition of one or more other features, integers, steps, operations, elements, components, and/or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

Exemplary embodiments of this disclosure are described herein with reference to illustrations of idealized embodiments (and intermediate structures) of this disclosure. As such, variations from various illustrated shapes as a result, for example, of manufacturing techniques and/or tolerances, are to be expected. Thus, various exemplary embodiments of this disclosure should not be construed as necessarily limited to various particular shapes of regions illustrated herein, but are to include deviations in shapes that result, for example, from manufacturing.

Unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized and/or overly formal sense unless expressly so defined herein.

Furthermore, relative terms such as “below,” “lower,” “above,” and “upper” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below.

As used herein, a term “about” and/or “substantially” refers to a +/−10% variation from a nominal value/term. Such variation is always included in any given value/term provided herein, whether or not such variation is specifically referred thereto.

If any disclosures are incorporated herein by reference and such disclosures conflict in part and/or in whole with this disclosure, then to an extent of a conflict, if any, and/or a broader disclosure, and/or broader definition of terms, this disclosure controls. If such disclosures conflict in part and/or in whole with one another, then to an extent of a conflict, if any, a later-dated disclosure controls.

In some embodiments, various functions or acts can take place at a given location and/or in connection with the operation of one or more apparatuses or systems. In some embodiments, a portion of a given function or act can be performed at a first device or location, and a remainder of the function or act can be performed at one or more additional devices or locations.

The corresponding structures, materials, acts, and equivalents of all means or step plus function elements in the claims below are intended to include any structure, material, or act for performing the function in combination with other claimed elements as specifically claimed. The embodiments were chosen and described in order to best explain the principles of the disclosure and the practical application, and to enable others of ordinary skill in the art to understand the disclosure for various embodiments with various modifications as are suited to the particular use contemplated.

The diagrams, images and drawings depicted herein are illustrative. There can be many variations to the diagram or the steps (or operations) described therein without departing from the spirit of the disclosure. For instance, the steps can be performed in a differing order or steps can be added, deleted or modified. All of these variations are considered a part of the disclosure. It will be understood that those skilled in the art, both now and in the future, can make various improvements and enhancements which fall within the scope of the claims which follow.

The description of this disclosure has been presented for purposes of illustration and description, but is not intended to be fully exhaustive and/or limited to the disclosure in the form disclosed. Many modifications and variations in techniques and structures will be apparent to those of ordinary skill in an art without departing from a scope and spirit of this disclosure as set forth in the claims that follow. Accordingly, such modifications and variations are contemplated as being a part of this disclosure. A scope of this disclosure is defined by various claims, which include known equivalents and unforeseeable equivalents at a time of filing of this disclosure.

1. A device comprising:
a transistor comprising a graphene-based conducting channel; and
a functional layer modified with an aptamer, and attached to the graphene-based conducting channel via a polymer,
wherein the aptamer binds with a target biomarker.

2. The device of claim 1, wherein the aptamer is an isolated enantiomer of D-ribose having SEQ ID NO:1.

3. The device of claim 1, wherein the polymer is a polyethylene glycol.

4. The device of claim 3, wherein a molecular weight of the PEG is from about 300 Da to about 3000 Da.

5. The device of claim 1, wherein the target biomarker is arginine vasopressin.

6. The device of claim 1, wherein a limit of detection of the device is from about 0.0001 pM to about 100 pM, where the limit of detection is a concentration of the target biomarker that is detectable by the device.

7. A method for manufacturing an aptamer-based biosensor comprising:
creating a graphene-based conducting channel in a field effect transistor;
coating the graphene-based conducting channel with a functional layer, wherein the functional layer is a polymer; and
reacting an aptamer with the functional layer to form the aptamer-based biosensor.

8. The method of claim 7, wherein the aptamer is an isolated enantiomer of D-ribose having SEQ ID NO:1.

9. The method of claim 7, wherein the polymer is a polyethylene glycol.

10. The method of claim 9, wherein a molecular weight of the PEG is from about 300 Da to about 3000 Da.

11. The method of claim 7, wherein the target biomarker is arginine vasopressin.

12. The method of claim 7, wherein a limit of detection of the device is from about 0.0001 pM to about 100 pM, where the limit of detection is a concentration of the target biomarker that is detectable by the device.

13. A method for detecting a target biomarker comprising:
manufacturing a device comprising a transistor with a graphene-based conducting channel;
coating the graphene-based conducting channel with a functional layer modified with an aptamer;
measuring a first drain-source current of the device;
exposing the device to a medium comprising a target biomarker;
measuring a second drain-source current of the device; and
determining binding of the aptamer to the target biomarker based on a difference between the first and second drain-source currents.

14. The method of claim 13, wherein the aptamer is an isolated enantiomer of D-ribose having SEQ ID NO:1.

15. The method of claim 13, wherein the polymer is a polyethylene glycol.

16. The method of claim 15, wherein a molecular weight of the PEG is from about 300 Da to about 3000 Da.

17. The method of claim 13, wherein the target biomarker is arginine vasopressin.

18. The method of claim 13, wherein a limit of detection of the device is from 0.0001 pM to 100 pM, where the limit of detection is a concentration of the target biomarker that is detectable by the device.

19. The method of claim 13, further comprising reversing a reaction of the aptamer with the functional layer.

20. The method of claim 7, further comprising reversing a reaction of the aptamer with the functional layer.


SEQUENCE LISTING (1 sequence)

SEQ ID NO: 1 - DNA, 66 bp, Synthetic construct
  source 1..66: mol_type = other DNA
ctctcgggacgacgtccaagtagtacgtttaattaggatttccgaattattggcatgcgtcgtccc